(19) United States
(12) Patent Application Publication
(10) Pub. No.: US 2014/0323859 A1
(43) Pub. Date: Oct. 30, 2014

(54) DEVICE AND PROCESS TO CONFIRM OCCLUSION OF THE FALLOPIAN TUBE

(71) Applicant: nVISION MEDICAL CORPORATION, Saratoga, CA (US)

(72) Inventor: Surbi Sarna, Saratoga, CA (US)

(21) Appl. No.: 14/357,875

(22) PCT Filed: Nov. 13, 2012
(86) PCT No.: PCT/US2012/064834

§ 371 Date: May 13, 2014

Related U.S. Application Data
(60) Provisional application No. 61/559,120, filed on Nov. 13, 2011.

(51) Int. Cl. A61B 5/00; A61B 8/12

(52) U.S. Cl. CPC A61B 5/4325; A61B 8/12; A61B 5/0073; A61B 5/0084
USPC 600/425; 600/463; 600/407; 600/478

(57) ABSTRACT

A device is provided to confirm intratubal occlusion in a subject of a fallopian tube having an inner diameter that includes a tubular shaft having a distal end and an interior lumen. An examination head is joined to the distal end of the shaft. A visualization modality in the examination head provides visual or acoustic imaging of the fallopian tube. A power source for the visualization modality is provided. A handle is provided for control of the device. An ex vivo imager of an ocular, video headgear, or a video display is provided in communication with the visualization modality. A process for evaluating an intratubal implant in a fallopian tube through optical or sonic wave visualization is also provided.

### CROSS-REFERENCE TO RELATED APPLICATIONS

This application is a non-provisional application that claims priority benefit of U.S. Provisional Application Ser. No. 61/559,120, filed Nov. 13, 2011 the contents of which are hereby incorporated by reference.

### FIELD OF THE INVENTION

The present invention in general relates to a medical device and process and system inclusive thereof, and in particular to a device and intratubal process for confirming the occlusion of a subject fallopian tube.

### BACKGROUND OF THE INVENTION

The ostium (plural ostia) of the fallopian tube may refer to the proximal or distal opening of the tube. The proximal tubal opening (ostium) is located within the uterus at the uterotubal junction and accessible via hysteroscopy. Occlusion at this opening is referred to as proximal tubal occlusion. The distal tubal opening (or abdominal ostium) is the opening in the infundibulum of uterine tube into the abdominal cavity. In ovulation, the oocyte enters the fallopian tube through this opening, and is surrounded by fimbriae, which help in the collection of the oocyte. Occlusion of the distal tubal opening is referred to as distal tubal occlusion.

Fallopian tube implants, often called intratubal implantations, induce reactions from the surrounding tissue, such as but not limited to inflammation and fibrosis. Following intratubal implantations, physicians need to determine the degree of tissue reaction and whether or not the tissue has responded to a desired degree to the implantation. In addition, they also need to check that the implant itself does not have any breach in integrity. Intratubal implants are used for medical purposes illustratively including sterilization or to treat hydrosalpinx.

Currently, the processs used to confirm intratubal implantations are the hysterosalpingogram (HSG), transvaginal, or pelvic, ultrasound. Confirmation of a successful intratubal implantation is usually performed three months after the implantation in the fallopian tube lumen in order to allow an appropriate tissue response.

However, there are several disadvantages to using the hysterosalpingogram. These limitations include the HSG requires radiology equipment and therefore most often is not done in the office of the gynecologist, and instead a patient is routinely sent to a separate radiology department to have the HSG procedure performed. Sending a patient out of the office is counterproductive in that a procedure conducted in office allows for better patient management, and increases the likelihood that the patient will return for a necessary follow-up procedure. Also, in while an in-office procedure increases patient convenience and reduces cost to both the healthcare system and to the patient; HSG is not amenable to these benefits. Furthermore, partly due to the fact that many HSG interpretative radiologists are not as familiar with the tortuosity and topography of the fallopian tube as reproductive specialists, HSG procedures have a high false negative rate of 30% (World Health Organization (WHO) (1986) “Comparative Trial Of Tubal Insufflation, Hysterosalpingography And Laparoscopy With Dye Hydrotubation For Assessment Of Tubal Patency” Fertil. Steril, 45, 1101-1107). In addition, patients often complain of pain and can be allergic to the dye used in the HSG procedure. HSG is also prone to spread infection through the dye infusion into the fallopian tube; as between 1-3% of all women who undergo HSG develop some type of infection soon after the procedure (Eric Daiter, MD Pelvic Factor Infertility: Uterine Cavity Infertility (2009)).

While pelvic ultrasound has been shown to successfully detect the placement of implantations consisting of metal coils (Kerin, F, Levy B S. “Ultrasound: An Effective Process For Localization Of The Echogenic Essure Sterilization Rnicro-Insert: Correlation With Radiologic Evaluations” J Minim. Invasive Gynecol 2005; 12: 50-54), ultrasound confirms only that the implantations exist within the patient and fails to provide information as to whether or not there has been a successful tissue reaction. Furthermore, transvaginal ultrasound can only be used on implants which are echogenic and therefore would not work on all materials used, such as implanted silicone matrices or to visualize tissue condition proximal to an implant.

Thus, there exists a need for a device and process to confirm intratubal implantations in a gynecologist office that is accurate and limits the aforementioned side effects to the patient. There further exists a need for a device and process that provides information as to the tissue reaction without resort to dye visualization.

### SUMMARY OF THE INVENTION

A device is provided to confirm intratubal occlusion in a subject of a fallopian tube having an inner diameter that includes a tubular shaft having a distal end and an interior lumen. An examination head is joined to the distal end of the shaft. A visualization modality in the examination head provides visual or acoustic imaging of the fallopian tube. A power source for the visualization modality is provided. A handle is provided for control of the device. An ex vivo imager of an ocular, video headgear, or a video display is provided in communication with the visualization modality.

A physician is able to determine the degree of tissue reaction and whether or not the implantation has been properly captured by the tissue. A device as provided may be used to guide the placement of an implantation. The integrity of the implant itself is also amenable to evaluation with the device detailed herein.

With resort to a working channel of a hysteroscope, or the working channel of other instruments access the ostia of the fallopian tubes within the uterus are also obtained. The process of visualization can also be completed with a catheter that places the intratubal implant in a subject. Visualization techniques operative herein include the use of fiber optics, sound waves for imaging, such as Intravascular Ultrasound (IVUS), and light scattering such as Optical Coherence Tomography (OCT). Images generated through the aforementioned visualization techniques are proved to a user or physician through an ocular, video headgear, or video display.

### BRIEF DESCRIPTION OF THE DRAWINGS

FIG. 1 illustrates a schematic side view of an inventive device with a portion depicted in partial cutaway;

FIG. 2A illustrates a view looking in to the distal end of the catheter of FIG. 1 with a transducer in place according to an embodiment invention;

FIG. 2B illustrates an inset to FIG. 2A depicting a manipulator extending from the operating channel of the device of FIG. 1;

FIG. 3A illustrates a cross-sectional view of an adjunct catheter device having a terminal transparent balloon associated with distal end, the balloon in fluid communication with a liquid reservoir and amenable to receiving a device per FIGS. 1 and 2 therein; and

FIG. 3B illustrates an expanding inset of the end of the catheter with a fluid filled balloon in a deployed position.

### DETAILED DESCRIPTION OF THE INVENTION

An inventive device and process is provided that has utility to confirm successful occlusion of the fallopian tube intratubal, including but not limited to purposeful occlusions caused by the implantation of polymer matrices such as silicones; or by the implantation of metal coils, such as coils made from nickel titanium alloy or stainless steel; or other implant materials, such as polyethylene terephthalate (PET), poly(ethylene oxide) (PEO) and poly(butylene terephthalate) copolymers (PBT), polyamides, or combinations thereof. Embodiments of the present invention allow a physician to determine the degree of tissue reaction and whether or not the implantation has been properly captured by the tissue, as well as the integrity of the implant itself. In specific embodiments of the present invention, an inventive device is used to guide the placement of an implantation.

The term “physician” is used herein to include all appropriate medical practitioners and explicitly inclusive of veterinarians, nurses, and technicians with an appropriately level of training Note that others with required training may be able to manipulate the inventive device.

The term “patient” is used herein synonymously with subject and is intended to include a female human primate, non-human primate, a horse, a cow, a sheep, a goat, a rabbit, a rat, a mouse, a cat, and a dog.

Among the non-limiting elements and features of embodiments of the invention to be discussed below are (a) image capture through various visualization process within a catheter, (b) compatibility with a hysteroscope or the catheters used to place implantations within the fallopian tubes, (c) a handle for manipulating the inventive device, and (d) procedures with the inventive device are suitable to be conducted in-office by the gynecologist thereby eliminating the need for a radiologist.

Embodiments of the invention may be used through a working channel of a conventional hysteroscope or the working channel of other devices which can be used to access the ostia of the fallopian tubes within the uterus. Hysteroscopies are conducted by reproductive specialists, commonly in-office, to visualize and determine the health of the uterine cavity. In the last ten years, less invasive sterilization procedures have also used the hysteroscope to locate the ostia of the fallopian tubes.

In embodiments of the invention used in conjunction with a hysteroscope, once the hysteroscope has located the ostia, a guidewire is placed through the hysteroscope and into the fallopian tubes. Then, another catheter, which acts as a sheath, is placed over the guidewire. The guidewire is then removed. A form of visualization component, as described further below, is then placed in the guidewire lumen of the catheter. Embodiments of the invention may be used to access the entire length of the fallopian tube.

In another inventive embodiment, since intratubal implants are usually only a short distance in from the ostia, typically one to three centimeters, there is no need for a guidewire and sheath, and the guidewire and sheath are eliminated. Instead, navigational functionality is combined with a visualization component to form a single catheter. Eliminating the need for the guidewire has the advantage of reducing the number of steps required by the physician, therefore reducing complexity and shortening overall procedural time.

In still other inventive embodiments are located on a catheter which places the intratubal implant in a patient. It is appreciated that embodiments of the present invention are operative both with or without a guidewire.

Visualization techniques used in embodiments of the invention illustratively include fiber optics; sound waves for imaging, such as Intravascular Ultrasound (IVUS) and photoacoustic imaging, and light scattering such as Optical Coherence Tomography (OCT) and polarized light scattering. Images generated through the aforementioned visualization techniques are transmitted to a physician directly through an eyepiece or video display; or wirelessly to a video display with a transceiver for wireless reception of the imaging signal.

The fiber optic visualization technique used in embodiments of the invention utilizes fiber optic bundles, as in the form of an endoscope. A catheter containing such fiber optics is compatible with pre-existing capital equipment found in the office of a gynecologist or other physician. However, a conventional fiber optic based endoscope only allows the physician to view the portion of the implant facing the uterus and does not supply information regarding tissue growth surrounding other portions of the implant.

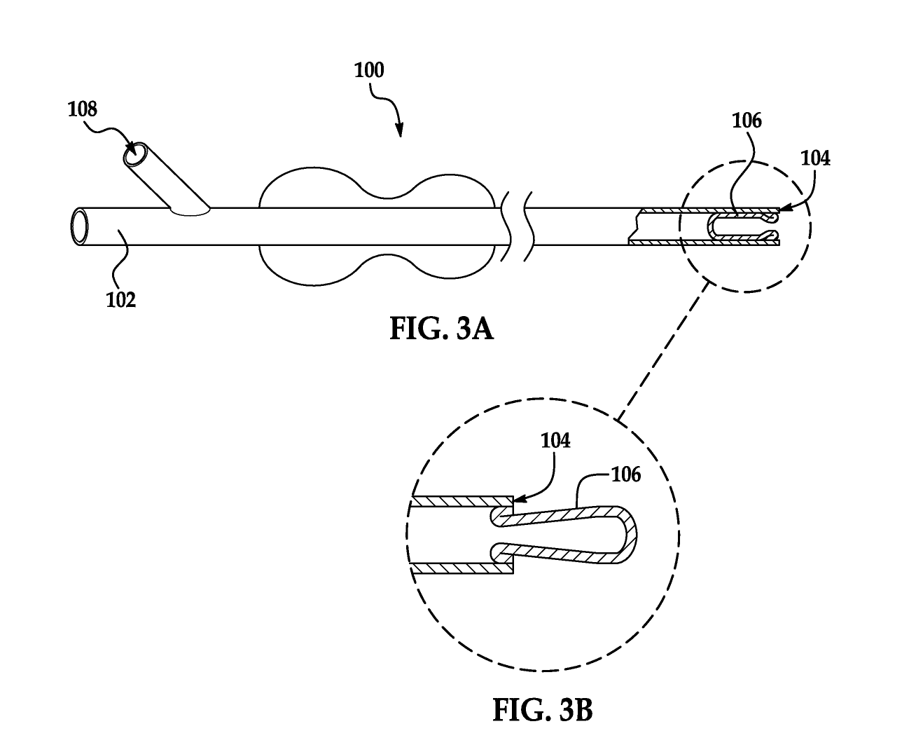

In an embodiment of the present invention utilizing ultrasound visualization, the space between the distal end of the catheter that contains the ultrasound transducer and the intratubal implant is filled with liquid, while in another embodiment, an optically or sonically transparent liquid-filled balloon, as shown in FIGS. 3A and 3B is deployed. An example of a suitable liquid is saline. In one mode of operating the inventive device, similar to selective salpingography (SSG), the portion of the fallopian tubes nearest the uterus is blocked in order to contain the liquid. Using the ultrasound-on-catheter process, both the implant and the surrounding tissue are visualized. The comprehensive visualization provided by the ultrasound-on-catheter process enables the physician to determine both whether or not the implant has been properly seeded within the lumen/tissue and whether or not the tissue has yielded an adequate response to the implant. Specifically, the interaction between the tissue and the implant is visualized. Optical Coherence Tomography (OCT) allows the visualization of implants which are not echogenic and therefore cannot be seen by a standard pelvic ultrasound. OCT renders images based on the chemical composition of the implant material. For example, silicones have characteristic ratios of carbon, hydrogen and oxygen, and therefore present material specific OCT spectrographs. The rendering provided by OCT serve to both differentiate the implant from the surrounding subject tissue and to evaluate the tissue reactivity to the implant. In certain embodiments, the OCT technique allows the physician to evaluate tissue-implant interaction at a submicron resolution.

The aforementioned ultrasound and light-scattering techniques have an additional advantage over standard endoscopic techniques in that they allow for sub-surface imaging. In a case where only one surface of the implant is available, these two imaging modalities afford cross-sectional viewing of the entire implant, therefore rendering a three-dimensional image. For example, if the implant is formed of a silicone-matrix, OCT enables the physician to ensure the integrity of every portion of the implant and not just the available surface.

Regardless of the visualization technique used in embodiments of the invention, the inventive device provides the physician with the ability to maneuver an inventive device inside the body from outside the body with feedback provided via an eyepiece or video screen, and to inspect an intratubal implant. In an embodiment of the present invention utilizing fiber optics, the fiber bundle runs the entire length of the device. The distal end of the fiber is fixed to the distal end of the device, and utilizes known endoscopic optics to optimize the viewing capabilities of the device. The proximal end of the fiber is connected to a viewing apparatus. The distal end of the fiber in an embodiment exits the distal end of the device at its center (in order to provide an adequate amount of distance from the lumen of the fallopian tube and the camera). The fiber bundle diameter, in one embodiment, is in the range from 0.01 inches to 0.03 inches. For the embodiments utilizing visualization techniques which make use of light-scattering, such as OCT. or sound waves, such as IVUS, an appropriate transducer resides on the catheter, transmitting images.

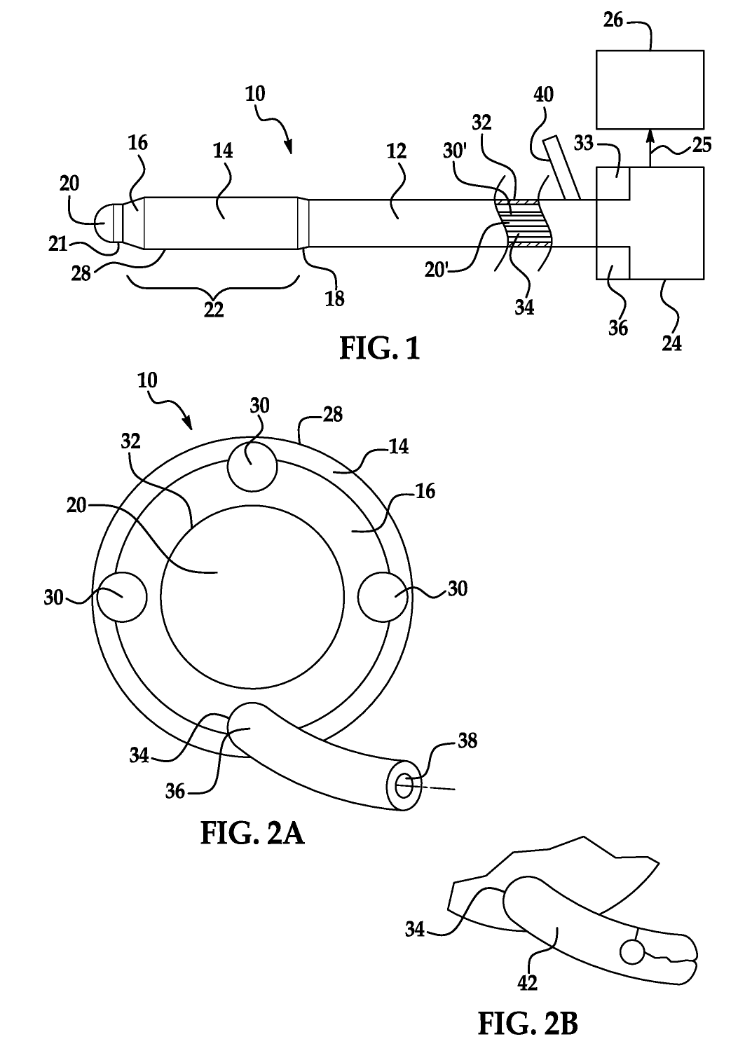

In yet another embodiment of the inventive device, instead of using a separate catheter, the transducers used for either OCT or IVUS are located directly onto catheters which are meant to be used to place the fallopian tube implant. FIGS. 1 and 2A show the isometric and cross-sectional view of the geometry of this embodiment.

Referring now to FIGS. 1 and 2A, an embodiment of the inventive device 10 configured as a catheter is shown. It is noted that drawing elements are not to scale. The inventive device 10 has a shaft 12 that has an outer diameter of less than or equal to 1.8 mm. Length of the shaft 12 is based on the length of the working channel of the hysteroscope plus the length of the fallopian tube or location of the implant. The length of the fallopian tube is, on average, ten cm and the implants are placed, on average, within two to three centimeters of the ostium. The examination head 14 of catheter shaft in this embodiment of the device has a larger outer diameter than the rest of the shaft 12. A fit is formed between the outer diameter surface 28 of the distal portion 22 of the examination head 14 of the device 10 and the inner diameter of the fallopian tube lumen of the patient being examined. The fit between the surface 28 and the fallopian tube lumen forms seal which can contain liquid between the examination head 14 of the device 10 and the implant to thereby aid in implant visualization, especially by sonic imaging. As described above a liquid is introduced in order to utilize intravascular ultrasound, where the space between the distal portion 22 of the device 10 that contains a visualization modality 20 in the form of an ultrasound transducer and the implant is liquid-filled. A rear taper transition 18 between the examination head 14 of the device 10 and the remaining part of the shaft 12 is provided in order to prevent sharp edges from hitting and potentially tearing the patient's body tissue upon extraction of the device 10. Similarly, a front taper 16 is provided to examination head 14 to prevent sharp edges from hitting and potentially tearing the patient's body tissue upon introduction of the device 10. The front taper 16 extends from examination head 14 to the front tip 21 of the device 10 which is also the exposed lip of the inner lumen 32. The outer layer 28 of the device 10 has a macroscopically smooth surface which eases navigation within the patient. The outer layer 28 in some embodiments is coated with lubricating substances. The interior lumen 32 of the device 10 either holds the visualization modality 20 in place or creates a working channel through which the visualization modality 20 is placed. As described above, the visualization modality 20 may be a transducer of sound-wave or light-scattering technology, or the distal end of a fiber optic bundle. The visualization modality 20 is in communication with a power source 33 and imager 26 by leads 20′. In certain embodiments, a device 10 has at least one fiber end 30 that provides additional lighting and extend via fiber 30′ to a light source 35, and the fiber end 30 is positioned on the front tip 21 of the device 10. The lighting would be needed if the imaging modality is fiber optics. A working channel 34 for an optional guide wire 36 (here, the guide wire is present) is provided in the device 10. It is appreciated that a guide wire is certain embodiments has a hollow channel 38 in fluid communication with a liquid reservoir 40. Alternatively, a manipulator 42 such as that detailed in U.S. Pat. No. 4,880,015 extends from working channel 34 as shown at 42 in the inset of FIG. 2. A manipulator 42 afford a physician the ability to adjust the visualized environment and illustratively is used to reposition the intratubal implant, reposition tissue precluding the formation of a seal between the distal portion 22 and the fallopian tube, or to collect a biopsy of tissue for ex vivo pathology study.

The handle 24 of the inventive device 10 is at the proximal end of the device 10, external to the patient's body. The handle 24 provides the physician with the capabilities of both steering the device 10 during insertion and use and provides the support and termination of all external connections to the inventive device 10. The handle 24 may have a wired or wireless connection, as represented by arrow 25, to an imager 26. The imager 26 may be a display, eyepiece, or video headgear.

In certain embodiments of the present invention a device 10, is fed into a catheter shown generally at 100 in FIG. 3. The catheter 100 has a bore 102 that is proportioned to receive a device 10 such that the distal end 22 of device 10 per FIGS. 1 and 2 extends to the end 104 of the catheter 100. The end 104 terminates in a sonically transparent balloon 106 that is has an undeployed position and a deployed position, with fluid pressure within the bore 102 controlling the deployment position of the balloon 106. The balloon is shown in FIG. 3B in a deployed position. In certain embodiments the balloon is dimensioned to deploy within the fallopian tube. A balloon 106 is readily formed of silicone and joined to that end 104 by conventional techniques including sonic welding and contact adhesives. The catheter 100 has a portal 108 for controlling the delivery of pressurizing fluid, such as saline solution into the bore 102. A separation handle 110 is provided in some embodiments of the catheter 100 to promote insertion into proximity to the fallopian tube of a subject.

References cited in the application are incorporated by reference to the same extent as if each reference was individual and explicitly incorporated by reference. These references are consistent with the state of the art in the field of the invention at the time the present application is filed.

The foregoing description is illustrative of particular embodiments of the invention, but is not meant to be a limitation upon the practice thereof. The following claims, including all equivalents thereof, are intended to define the scope of the invention.

1. A device to confirm intratubal occlusion in a subject of a fallopian tube having an inner diameter, said device comprising:
a tubular shaft having a distal end and an interior lumen;
an examination head joined to the distal end of said shaft;
a visualization modality in said examination head for visual or acoustic imaging of the fallopian tube;
a power source for said visualization modality;
a handle for control of said device and for support; and
an ex vivo imager of an ocular, video headgear, or a video display in communication with said visualization modality.

2. The device of claim 1 wherein said tubular shaft has a first outer diameter and said examination head has a second outer diameter that is greater than the first outer diameter designed to fit against the inner diameter of the fallopian tube lumen wherein said first outer diameter is 1.8 mm.

3. The device of claim 2 further comprising a rear taper transition between the first outer diameter and the second outer diameter.

4. The device of claim 1 further comprising a front taper transition extending from said examination head to a front tip of said examination head.

5. The device of claim 1 wherein said smooth outer surface is coated with a lubricating substance.

6. The device of claim 1 wherein said visualization modality includes an ultrasonic transducer.

7. The device of claim 1 wherein said visualization modality includes an ultrasonic transducer and further comprising a reservoir of fluid for delivering said fluid to a volume between said ultrasound transducer and the inner diameter of the fallopian tube.

8. The device of claim 1 wherein said imager is in wireless communication with said visualization modality.

9. The device of claim 1 wherein said visualization modality is visual and a fiber optic providing an image to said imager through the interior lumen of said shaft.

10. The device of claim 9 further comprising at least one fiber end terminating in said examination head and coupled to a light source for lighting the fallopian tube.

11. The device of claim 1 wherein said visualization modality is Intravascular Ultrasound (IVUS).

12. The device of claim 1 further comprising a working channel extending through the interior lumen to said examination head.

13. The device of claim 12 further comprising one of a solid guide wire, hollow guide wire, or a manipulator sliding within said working channel to extend from said examination head.

14. The device of claim 1 wherein said visualization modality is optical and is Optical Coherence Tomography (OCT).

15. The device of claim 1 further comprising a catheter having an end and a bore proportioned to receive said shaft such that said examination head extends to the end of said catheter, the end terminates in a sonically transparent balloon adapted to insert with the inner diameter of the fallopian tube.

16. The device of claim 15 wherein said shaft is configured to slide in the working channel of a hysteroscope.

17. A process of evaluating an intratubal implant in a fallopian tube comprising:
inserting the device according to claim 1 into proximity to the fallopian tube; and
visualizing the intratubal implant.
